(19) United States
(12) Patent Application Publication
(10) Pub. No.: US 2016/0143958 A1
(43) Pub. Date: May 26, 2016

(54) METHOD OF TREATING VIRAL DISEASES

(71) Applicant: Robert Bayer, Orono, ME (US)

(72) Inventor: Robert Bayer, Orono, ME (US)

(21) Appl. No.: 14/948,338

(22) Filed: Nov. 22, 2015

Related U.S. Application Data
(60) Provisional application No. 62/083,228, filed on Nov. 22, 2014.

(51) Int. Cl. A61K 35/612; A61K 38/17

(52) U.S. Cl. CPC A61K 35/612; A61K 38/1767

(57) ABSTRACT

Lobster hemolymph and compositions derived therefrom for the prevention or treatment of viral diseases by systemic administration.

### CROSS-REFERENCE TO RELATED APPLICATIONS

This application claims the benefit of co-pending US Provisional Patent Application Ser. No. 62/083,228, filed 22 Nov. 2014, which is hereby incorporated herein as though fully set forth.

### FIELD OF INVENTION

This invention is in the field of treatment of viral diseases by internally administering to a patient lobster hemolymph or compositions derived therefrom.

### BACKGROUND

Lobster (Homarus americanus) hemolymph is a readily available byproduct of lobster processing that is currently discarded as waste.

There are approximately 215,000,000 pounds of lobster landed annually between the U.S. and Canada. We estimate that close to 60,000,000 pounds is processed by the food industry. The hemolymph from processed lobsters is currently discarded at an estimated rate of 2 million pounds per year.

Homarus americanus as a source of hemolymph is unique because lobster hemolymph/hemocyanin is readily available in sufficient volume to support its widespread use. This is not the case with hemolymph from other crustaceans and molluscs. Unlike shrimp, oysters, clams and other shellfish, lobsters contain a much larger volume of hemolymph—approximately 100 times more by volume. Additionally, unlike other shellfish, extraction of lobster hemolymph is carried out during typical lobster processing. Though it is currently discarded, it can be harvested and utilized in our invention.

### SUMMARY OF INVENTION

Our invention is a new utilization and a value-added product for the lobster industry. The invention provides for a formulation and process to utilize hemolymph, or its component hemocyanin, from the lobster (Homarus americanus, Homarus gammarus, and Panulirus argus) for the prevention or treatment of viral infections.

In an illustrative embodiment, the method of the invention comprises internally administering to a mammal suffering from a viral infection a pharmaceutical composition that is made from lobster hemolymph or its extracted hemocyanin (either in a natural state or a derivatized state) in combination with a pharmaceutically acceptable excipient, or as an adjuvant to an antiviral therapy.

In illustrative embodiments, the anti-viral properties of lobster hemocyanin are enhanced by fractionation and/or glycosylation.

In illustrative embodiments, the inventions comprises a pharmaceutical composition formulated for internal administration comprising as an active pharmaceutical ingredient (i) lobster hemolymph or lobster hemocyanins, (ii) an active fraction of lobster hemocyanins, or (iii) a derivatized form of lobster hemocyanins or active fractions of such derivatized forms.

In illustrative embodiments, lobster hemolymph, lobster hemocyanin, or a derivative of lobster hemocyanin is employed as an adjuvant, i.e., an agent that stimulates the immune system of a mammal or is employed as an antiviral, i.e., an agent that has viracidal activity, or as both an immune system stimulator and a viracide.

### BRIEF DESCRIPTION OF THE DRAWINGS

These and other features of this invention will be more readily understood from the following detailed description of the various aspects of the invention taken in conjunction with the accompanying drawings that depict various embodiments of the invention, in which:

FIG. 1 shows a graph of the strength of response to rabbit-sourced Troponin C protein;

FIG. 2 shows a graph of the strength of response to keyhole limpet hemocyanin; and

FIG. 3 shows a graph of the response to lobster hemolymph.

The drawings are intended to depict only typical aspects of the invention, and therefore should not be considered as limiting the scope of the invention. In the drawings, like numbering represents like elements among the drawings.

### DETAILED DESCRIPTION OF INVENTION

This invention utilizes lobster hemolymph/hemocyanin as an anti-viral treatment of disease like ebola, herpes, polio, HIV, Influenza (e.g., H1N1), measles, and others.

The hemolymph can be used as extracted from lobster or it can be diluted with a liquid carrier. Alternatively, the hemocyanin can be obtained in purified or partially purified form from lobster hemolymph by standard purification techniques including, e.g., centrifugation and chromatography. See, e.g., Bolton et al. (2014).

Derivatives of lobster hemolymph useful in the practice of this invention include, e.g., fragmented lobster hemocyanin, an active fraction of lobster hemocyanin or of fragmented hemocyanin, or a glycosylated form of lobster hemocyanin or of fragmented lobster hemocyanin.

Fragmentation by proteolytic enzymes along with glycosylation also allows for recombination into larger molecules that are more active and stable. Lobster hemocyanin has increased activity following fragmentation with proteolytic enzymes or sonication. Fragmented hemocyanin can be prepared by admixing and reacting a protease with lobster hemocyanin for a time and under conditions such that the hemocyanin is proteolyzed. Proteolytic enzymes include but are not limited to subtilisins. Thus, derivatives of lobster hemolymph useful in the practice of the invention can also be prepared by fragmenting the hemocyanin.

Active fractions of lobster hemocyanin are fractions of the hemocyanin or of fragmented hemocyanin that stimulate the immune system and induce or enhance the immune response to a virus with which the mammal is infected or may become infected and/or have direct antiviral activity. Derivatives of lobster hemolymph comprising such active fractions can be prepared such as by separating the component hexamers and dodecamers, e.g., as described by Bolton et al. (2014).

Hemocyanin-enriched fractions of hemolymph can be prepared, e.g., by low speed centrifugation of whole hemolymph to remove cells and collecting the supernatant. Hemocyanins can be further purified such as by high speed centrifugation ion exchange chromatography, and/or dialysis. In an illustrative embodiment, a hemocyanin enriched fraction is prepared by allowing the hemolymph to stand whereby a precipitate is formed and the supernatant can be decanted and then subjected to further purification, e.g., by micofiltration.

Adding sugars, including but not limited to sucrose, fructose, and trehalose to lobster hemocyanin enhances stability and can also enhance the immune stimulating and/or antiviral activity of the hemocyanin. See, e.g., Zanjani, et al. (2014). A similar effect can be obtained by mixing the hemocyanin or a derivative thereof with honey, e.g., from Apis mellifera. The hemocyanin that has been mixed with or otherwise treated with one or more sugars, including by admixing with honey, are herein referred to as “glycosylated forms” of lobster hemocyanin or of derivatives thereof.

A mammal treated in accordance with this invention can be, e.g., a farm animal such as a pig, sheep, horse, or cow, a companion animal such as a dog, cat, rabbit, or ferret, or a primate such as a monkey, ape, or human being.

Derivatives of lobster hemocyanin suitable for use as adjuvants or for the prophylaxis or treatment of viral infections in accordance with embodiments of the invention may be selected from the group consisting of a hexamers, dodecamers, and polypeptide subunits of the hemocyanin, active fragments of such multimers and polypeptides, and synthetically glycosylated forms of the polypeptides present in the hemocyanin, active fractions, or active fragments. Suitable hemocyanin derivatives for use in an embodiment of the invention can be identified such as by antiviral assays based on cell viability. For example, a cell line infected with the virus of interest or which is exposed to the virus can be treated with hemocyanin or a derivative thereof, and the efficacy of the treatment evaluated. Derivatives of hemocyanin used in methods and compositions embodied by the invention will typically have substantially the same antiviral activity as the intact hemocyanin or the antiviral activity will be enhanced generally or with respect to a particular virus or group of related viruses.

The hemocyanin may be administered for prophylaxis or treatment of infection, physical manifestations and/or symptoms by a virus selected from, but not limited to, the group consisting of ebola viruses, Herpes viruses and in particular Herpes Simplex viruses (e.g., HSV-1 (predominantly oral) and HSV-2 (predominantly genital)), Herpes Zoster (VZV), Equine Herpesvirus-1 (EHV-1), Feline Herpesvirus-1 (FHV-1), Epstein-Barr virus (EBV), Human Immune Deficiency virus (HIV), Cytomegalovirus (CMV), human papilloma virus (HPV), rhinovirus, influenza virus (e.g., H1N1), and common cold viruses.

When used as an adjuvant, the hemocyanin or derivative thereof can be co-administered with the antigen(s) against which the immune response is to be directed. Such co-administration can comprise administration of a single pharmaceutical composition comprising both the hemocyanin or derivative thereof and the antigen(s) or in multiple compositions, simultaneously or sequentially. Co-administration can also be accomplished by coupling of the hemocyanin or derivative to the antigen(s).

The antigen(s) can be any molecule to which an immune response can be generated. The antigen can, for example, be a viral, bacterial or other microbial antigen. Typically, the antigen will be a viral antigen such as an outer membrane protein or other suitable antigen. The antigen can be a whole molecule or a fragment thereof containing an epitope presented by the intact pathogen.

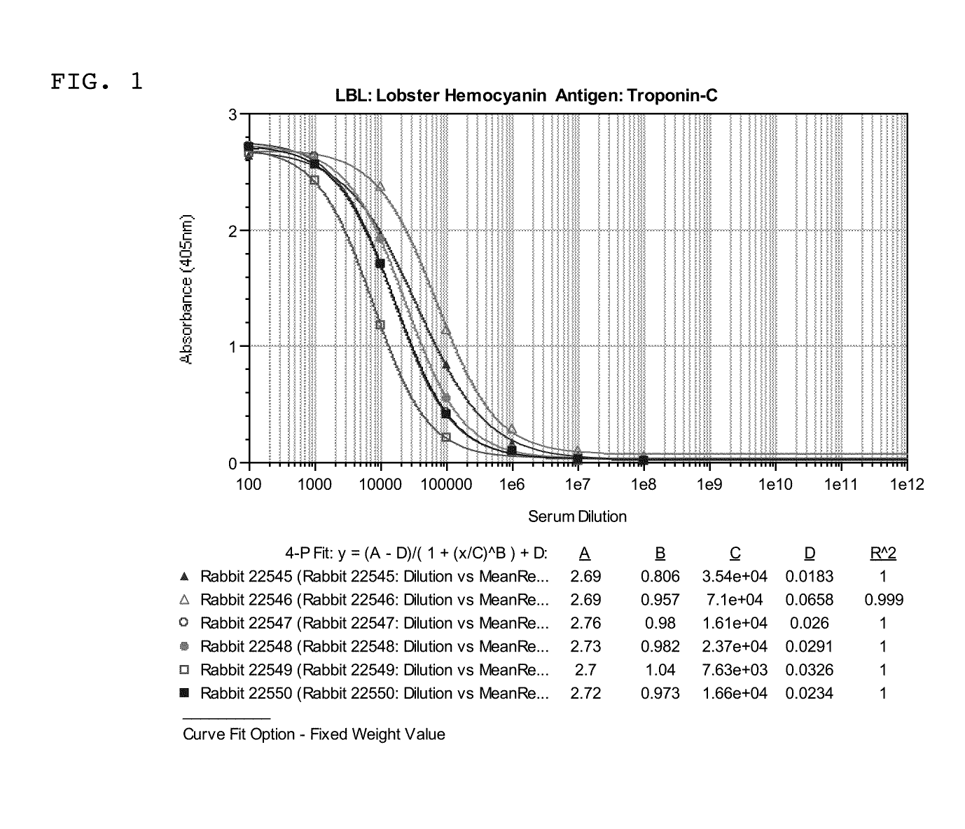

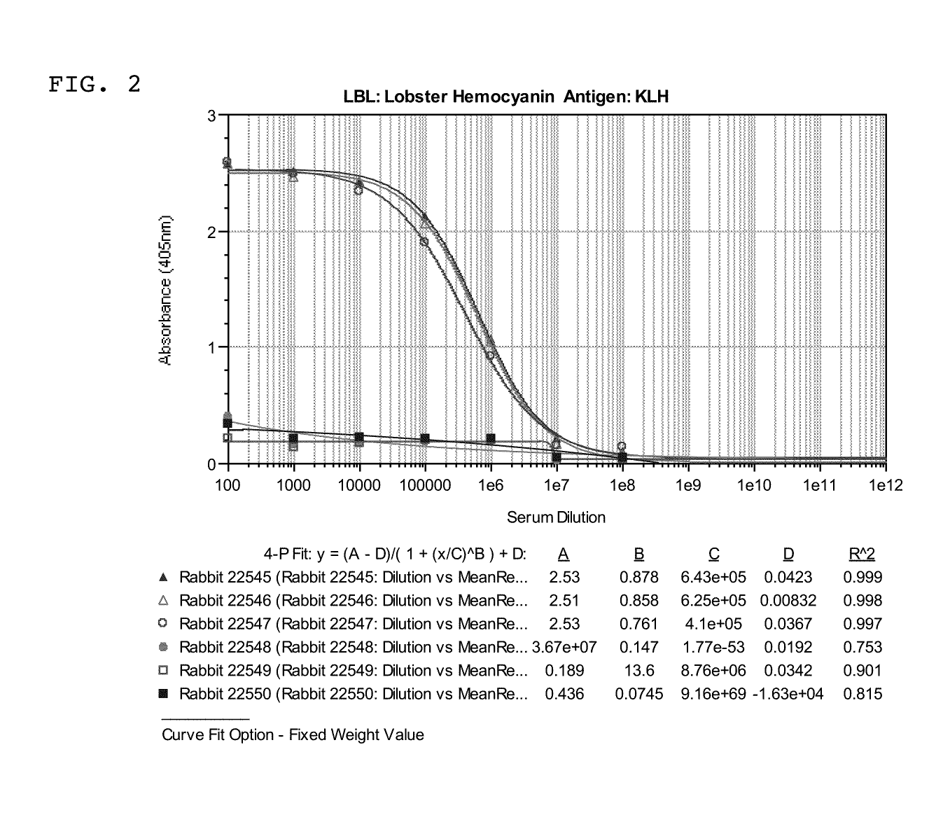

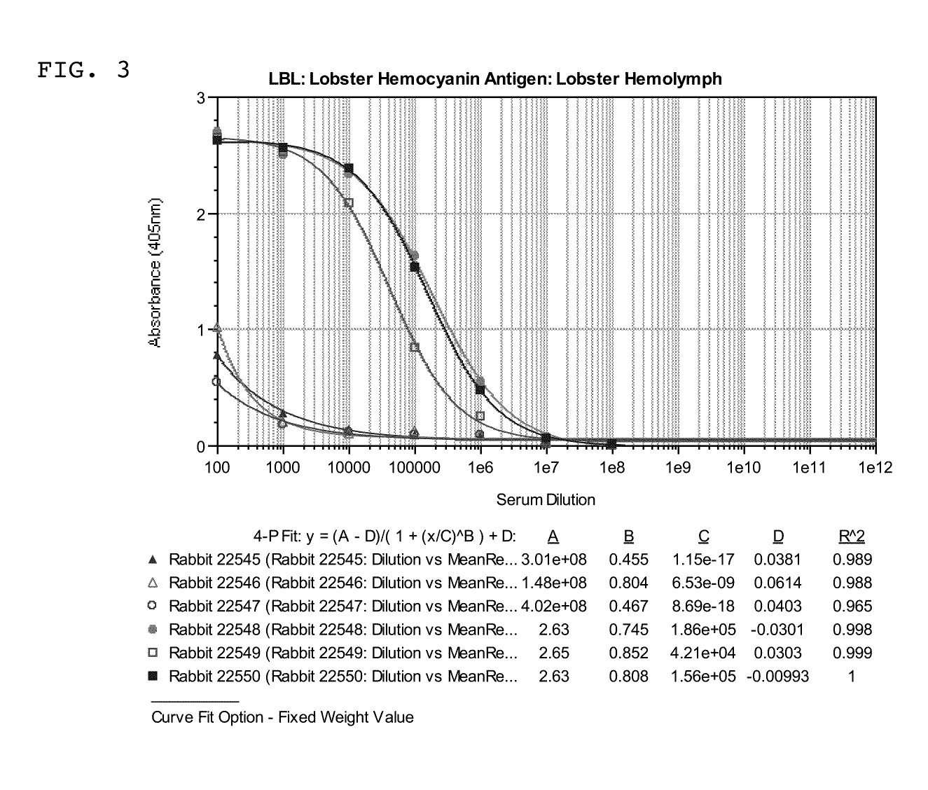

FIGS. 1-3, for example, show graphs of the strengths of response to three antigens—rabbit-sourced Troponin C (FIG. 1), keyhole limpet hemocyanin (KLH; FIG. 2), and lobster hemolymph (LH; FIG. 3).

The lobster hemocyanin or derivative thereof, which can be administered with or without a adjuvant and with or without an antigen, is administered internally. In illustrative embodiments, the lobster hemocyanin or derivative thereof, with or without an adjuvant or an antigen, is delivered systemically. Suitable routes of administration include, e.g., oral, transdermal, transmucosal (e.g., sublingual), and parenteral (e.g., subcutaneous, intramuscular, intravenous, and intrathecal).

It will be understood that the amount of the composition actually administered will be determined by a physician, in the light of the relevant circumstances including the condition to be treated, the choice of composition to be administered, the chosen route of administration, the age, weight, and response of the individual patient, and the severity of the patient's symptoms.

For therapeutic or prophylactic use, lobster hemolymph or lobster hemocyanin or a derivative thereof as described above will normally be administered as a pharmaceutical composition comprising as the (or an) essential active ingredient the lobster hemolymph or lobster hemocyanin or a derivative thereof in association with a solid or liquid pharmaceutically acceptable carrier and, optionally, with pharmaceutically acceptable adjuvants and excipients employing standard and conventional techniques.

The phrase “pharmaceutical composition” refers to a composition suitable for administration in medical (or veterinary) use. It should be appreciated that the determinations of proper dosage forms, dosage amounts, and routes of administration for a particular patient are within the level of ordinary skill in the pharmaceutical and medical or veterinary arts. The pharmaceutical compositions include suitable dosage forms for oral, parenteral (including subcutaneous, intramuscular, intradermal, intravenous, transdermal (such as via a dermal patch, gel, microneedle, iontophoresis, sonophoresis, or phonophoresis), bronchial or nasal administration. Thus, if a solid carrier is used, the preparation may be tableted, placed in a hard gelatin capsule in powder or pellet form, or in the form of a troche or lozenge. The solid carrier may contain conventional excipients such as binding agents, fillers, tableting lubricants, disintegrants, wetting agents and the like. The tablet may, if desired, be film coated by conventional techniques. If a liquid carrier is employed, the preparation may be in the form of a syrup, emulsion, soft gelatin capsule, sterile vehicle for injection, an aqueous or non-aqueous liquid suspension, or may be a dry product, e.g., a lyophilisate, for reconstitution with water or other suitable vehicle before use. Liquid preparations may contain conventional additives such as suspending agents, emulsifying agents, wetting agents, non-aqueous vehicle (including edible oils), preservatives, as well as flavoring and/or coloring agents. For parenteral administration, a vehicle normally will comprise sterile water, at least in large part, although saline solutions, glucose solutions and the like may be utilized. Injectable suspensions also may be used, in which case conventional suspending agents may be employed. Conventional preservatives, buffering agents and the like also may be added to the parenteral dosage forms. See, for example, Remington's Pharmaceutical Sciences, Mack Publishing Company, Easton, Pa., 17th edition, 1985. For parenteral administration, the hemolymph, hemocyanin, or a derivative of hemocyanin would be sterilized, e.g., by filtration of a hemocyanin-enriched fraction of hemolymph or cell-free hemocyanin or hemocyanin derivative.

In making pharmaceutical compositions containing lobster hemolymph or lobster hemocyanin or a derivative thereof as described hereinabove, the active ingredient(s) will usually be mixed with a pharmaceutically acceptable carrier, or diluted by a carrier, or enclosed within a carrier, which may be in the form of a capsule, sachet, paper, or other container. When the carrier serves as a diluent, it may be a solid, semi-solid, or liquid material which acts as a vehicle, excipient or medium for the active ingredient. Thus, the composition can be in the form of tablets, pills, powders, lozenges, sachets, cachets, elixirs, suspensions, emulsions, solutions, syrups, aerosols (as a solid or in a liquid medium), ointments containing for example up to 10% by weight of the active compound, soft and hard gelatin capsules, suppositories, sterile injectable solutions and sterile packaged powders.

The term “pharmaceutically acceptable” refers to compositions, carriers, diluents and reagents, that can be administered to a mammal, e.g., to a human being. Some examples of suitable carriers and diluents include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, syrup, methyl cellulose, methyl- and propylhydroxybenzoates, talc, magnesium stearate and mineral oil. The formulations can additionally include lubricating agents, wetting agents, emulsifying and suspending agents, preserving agents, sweetening agents, or flavoring agents. The compositions of the invention may be formulated so as to provide quick, sustained or delayed release of the active ingredient after administration to the patient.

Compositions suitable for parenteral administration conveniently comprise a sterile aqueous preparation of a compound or composition of the invention, which is preferably isotonic with the blood of the recipient. This aqueous preparation may be formulated according to known methods using suitable dispersing or wetting agents, emulsifying and suspending agents. Various antibacterial and antifungal agents, for example, parabens, chlorobutanol, phenol, and sorbic acid also may be included. The sterile injectable preparation also may be a sterile injectable solution or suspension in a non-toxic parenterally-acceptable diluent or solvent, for example, as a solution in 1,3-butane diol. Among the acceptable vehicles and solvents that may be employed are water, Ringer's solution, and isotonic sodium chloride solution. In addition, sterile, fixed oils are conventionally employed as a solvent or suspending medium. For this purpose any bland fixed oil may be employed including synthetic mono- or di-glycerides. In addition, fatty acids such as oleic acid may be used in the preparation of injectables. Prolonged absorption of the injectable pharmaceutical form can be brought about by the use of agents delaying absorption, for example, aluminum monostearate and gelatin. Carrier formulation suitable for subcutaneous, intravenous, intramuscular, etc. administrations can be found in Remington's Pharmaceutical Sciences, Mack Publishing Co., Easton, Pa.

The compositions are preferably formulated in a unit dosage form. The term “unit dosage form” refers to physically discrete units suitable as unitary dosages for human subjects and other mammals, each unit containing a predetermined quantity of active material calculated to produce the desired therapeutic effect, in association with the required pharmaceutical carrier. So, e.g., a pharmaceutical composition in intravenous unit dose form may comprise a vial or pre-filled syringe, each comprising an effective amount or a convenient fraction of an effective amount such that one the contents of one vial or syringe are administered at a time.

The present invention relates to a liquid, ointment, tablet, powder and/or crystals made from lobster hemolymph or its component hemocyanin (in its natural form and/or fractionated and glycosylated) for the treatment of viral infections and resulting lesions. The hemolymph is rendered particularly but not exclusively from the lobster genus: species Homarus americanus. Fractionation by proteolytic enzymes offers the possibility of recombination into larger molecules that are more active.

The invention also finds particular but not exclusive application for the treatment of viral diseases including but not limited to poliomyelitis (polio), HIV, Influenza (e.g., H1N1), and rubeola (measles).

It is understood that the examples and embodiments described herein are for illustrative purposes only and that various modifications or changes in light thereof will be suggested to persons skilled in the art and are to be included within the spirit and purview of this application and the scope of the appended claims.

All publications mentioned in this specification above or listed below are herein incorporated by reference as though fully set forth. Any discussion of documents, acts, materials, devices, articles or the like that has been included in this specification is solely for the purpose of providing a context for the present invention. It is not to be taken as an admission that any or all of these matters form part of the prior art base or were common general knowledge in the field relevant to the present invention as it existed anywhere before the priority date of this application.

### REFERENCE LIST

Bolton J, Bayer R, Bushway R, Collins S, Perkins B. 2014. Analytical and Semipreparative HPLC Analysis and Isolation of Hemocyanin from the American Lobster Homarus americanus. J Shellfish Res. 33(1):11-17.
Coates C and Nairn J. 2014. Developmental Comp Immunol. 2014. 45: 43-55.
Cuthbertson, Adrian. 2009. W02009129561 A1, also published as US 20110033499.
Dolashka P, Velkova L, Iliev I, Beck A, Dolashki A, Yossifova L, Toshkova R, Voelter W, Zacharieva S. 2003. Antitumor activity of glycosylated molluscan hemocyanins via Guerin ascites tumor. Eur Urol.;37 Suppl 3:34-40.
Velkova L, Dimitrov I, Schwarz H, Stevanovic S, Voelter W, Salvato B, Dolashka-Angelova P. 2010. Structure of hemocyanin from garden snail Helix lucorum. Comp Biochem Physiol. Part B 157: 16-25.
Dolashka P and Voelter W. 2013. Antiviral activity of hemocyanins. 2013. ISJ 10:120-127.
Greco K N, Mendonça R M, Moraes R H, Mancini D A, Mendonça R Z. 2004. Antiviral activity of the hemolymph of Lonomia obliqua (Lepidoptera: Saturniidae). Antiviral Res. Feb;61(2):93-9.
Olicard C, Renault T, Torhy C, Benmansour A, Bourgougnon N. 2005. Putative antiviral activity in hemolymph from adult Pacific oysters, Crassostrea gigas. Antiviral Res. Jun;66(2-3):147-52. Epub Apr 26.
Velkova, Lyudmila, Dimitrov, I., Schwarz, H., Stevanovic, S., Voelter, W., Salvato, B., Dolashka-Angelova, P. 2009. Structure of hemocyanin from garden snail Helix lucorum. Institute of Organic Chemistry, Bulgarian Academy of Sciences, Acad. G. Bonchev str b1.9, Sofia 1113, Bulgaria. Comparative biochemistry and physiology. Part B, Biochemistry & molecular biology (Impact Factor: 1.61). 04/2010; 157(1): 16-25. DOI: 10.1016/j.cbpb.2010.04.012 Source: PubMed.
Zanjani, Negar T., Fareed Sairi, Gavin Marshall, Monica Miranda Saksena, Peter Valtchev, Vincent G. Gomes, Anthony L. Cunningham, Fariba Dehghan. 2014. Formulation of abalone hemocyanin with high antiviral activity and stability. European Journal of Pharmaceutical Sciences, 53:77-85.
Zhang X, Huang C, Qin Q. 2003. Antiviral properties of hemocyanin isolated from shrimp Penaeus monodon. (Key Laboratory of Marine Biogenetic Resources, The Third Institute of Oceanography, State Oceanic Administration, 361005, Xiamen, PR China.)

1. A method of treating a viral infection in a mammal afflicted therewith, said method comprising internally administering an effective amount of a lobster hemolymph, lobster hemocyanin, or a derivative of lobster hemocyanin.

2. The method of claim 1 wherein the derivative of lobster hemocyanin is selected from fragmented lobster hemocyanin, an active fraction of lobster hemocyanin or of fragmented hemocyanin, or a glycosylated form of lobster hemocyanin or of fragmented lobster hemocyanin.

3. The method of claim 1 or 2 wherein the lobster hemocyanin or derivative thereof is administered orally, transdermally, transmucosolly, or parenterally.

4. The method of claim 2 or 3 wherein the hemocyanin preparation is proteolyzed lobster hemocyanins prepared by admixing and reacting a protease with lobster hemocyanins for a time and under conditions such that the hemocyanins are proteolyzed.

5. The method of any of the preceding claims wherein the lobster is Homarus americanus, Homarus gammarus, or Panulirus argus.

6. The method of any of the preceding claims wherein the hemocyanin or derivative thereof is administered in a pharmaceutical composition comprising one or more sugars.

7. The method of claim 6 wherein the composition comprises honey.

8. A pharmaceutical composition for internal administration to a mammal for the prevention or treatment of a viral infection in the mammal, said composition comprising lobster hemolymph, lobster hemocyanin, or a derivative of lobster hemocyanin.

9. The composition of claim 8 wherein the derivative of lobster hemocyanin is selected from fragmented lobster hemocyanin, an active fraction of lobster hemocyanin or of fragmented hemocyanin, or a glycosylated form of lobster hemocyanin or of fragmented lobster hemocyanin.

10. The composition of claim 9 comprising one or more sugars.

11. The composition of claim 9 comprising honey.
